(19) United States
(12) Patent Application Publication
(10) Pub. No.: US 2016/0038315 A1
(43) Pub. Date: Feb. 11, 2016

(54) TREATMENT OF CARDIAC DISEASES THAT PROMOTE EPICARDIAL ARTERY ENLARGEMENT WITH BIORESORBABLE SCAFFOLDS

(71) Applicant: Abbott Cardiovascular Systems Inc., Santa Clara, CA (US)

(72) Inventors: Paul Consigny, San Jose, CA (US); Alexander J. Sheehy, Redwood City, CA (US)

(21) Appl. No.: 14/454,595

(22) Filed: Aug. 7, 2014

(51) Int. Cl. A61F 2/82

(52) U.S. Cl. CPC A61F 2/82

(57) ABSTRACT

Methods of treating coronary artery disease (CAD) and a cardiac disease that stimulates enlargement of the epicardial artery with a bioresorbable scaffold in a patient in need thereof are disclosed. Methods of treating CAD and an event that precipitates enlargement of the epicardial artery with a bioresorbable scaffold in a patient in need thereof are disclosed.

### FIELD OF THE INVENTION

This invention relates to bioresorbable polymer scaffolds and methods of treatment of coronary artery disease and cardiac diseases that stimulate, permit, or enable, enlargement of epicardial arteries with bioresorbable scaffolds.

### DESCRIPTION OF THE STATE OF THE ART

This invention relates generally to methods of treatment with radially expandable endoprostheses that are adapted to be implanted in a bodily lumen. An “endoprosthesis” corresponds to an artificial device that is placed inside the body. A “lumen” refers to a cavity of a tubular organ such as a blood vessel. A stent is an example of such an endoprosthesis. Stents are generally cylindrically shaped devices that function to hold open and sometimes expand a segment of a blood vessel or other anatomical lumen such as urinary tracts and bile ducts. Stents are often used in the treatment of atherosclerotic stenosis in blood vessels. “Stenosis” refers to a narrowing or constriction of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

Stents are typically composed of a scaffold or scaffolding that includes a pattern or network of interconnecting structural elements or struts, formed from wires, tubes, or sheets of material rolled into a cylindrical shape. This scaffold gets its name because it physically holds open and, if desired, expands the wall of a passageway in a patient. Typically, stents are capable of being compressed or crimped onto a catheter so that they can be delivered to and deployed at a treatment site.

Delivery includes inserting the stent through small lumens using a catheter and transporting it to the treatment site. Deployment includes expanding the stent to a larger diameter once it is at the desired location. Mechanical intervention with stents has reduced the rate of restenosis as compared to balloon angioplasty.

Stents are used not only for mechanical intervention but also as vehicles for providing biological therapy. Biological therapy uses medicated stents to locally administer a therapeutic substance. The therapeutic substance can also mitigate an adverse biological response to the presence of the stent. A medicated stent may be fabricated by coating the surface of either a metallic or polymeric scaffolding with a polymeric carrier that includes an active or bioactive agent or drug. Polymeric scaffolding may also serve as a carrier of an active agent or drug.

The stent must be able to satisfy a number of mechanical requirements. The stent must have sufficient radial strength so that it is capable of withstanding the structural loads, namely radial compressive forces imposed on the stent as it supports the walls of a vessel. Radial strength, which is the ability of a stent to resist radial compressive forces, relates to a stent's radial yield strength and radial stiffness around a circumferential direction of the stent. A stent's “radial yield strength” or “radial strength” (for purposes of this application) may be understood as the compressive loading, which if exceeded, creates a yield stress condition resulting in the stent diameter not returning to its unloaded diameter, i.e., there is irrecoverable deformation of the stent. When the radial yield strength is exceeded the stent is expected to yield more severely and only a minimal force is required to cause major deformation.

Once expanded, the stent must adequately provide lumen support during a time required for treatment in spite of the various forces that may come to bear on it, including the cyclic loading induced by the beating heart. In addition, the stent must possess sufficient flexibility with a certain resistance to fracture.

Stents made from biostable or non-degradable materials, such as metals that do not corrode or have minimal corrosion during a patient's lifetime, have become the standard of care for percutaneous coronary intervention (PCI) as well as in peripheral applications, such as the superficial femoral artery (SFA). Such stents have been shown to be capable of preventing early and later recoil and restenosis. A stents with a non-biodegradable metallic body is referred to as bare metal stent (BMS). A drug eluting stent (DES) refers to a stent with non-biodegradable or durable metallic body that includes a therapeutic coating. The coating can include a polymer and a drug. The polymer functions as a drug reservoir for delivery of the drug to a vessel. The polymer can be non-biodegradable or bioresorbable.

In order to affect healing of a diseased blood vessel, the presence of the stent is necessary only for a limited period of time, as the artery undergoes physiological remodeling over time after deployment. The development of a bioabsorbable stent or scaffold could obviate the permanent metal implant in vessel, allow late expansive luminal and vessel remodeling, and leave only healed native vessel tissue after the full resorption of the scaffold. A stent or scaffold with a bioabsorbable body may be referred to as a bioabsorbable vascular scaffold (BVS). Stents fabricated from bioresorbable, biodegradable, bioabsorbable, and/or bioerodable materials such as bioabsorbable polymers can be designed to completely absorb only after or some time after the clinical need for them has ended.

### SUMMARY OF THE INVENTION

A first embodiment of the present invention includes a method of treating coronary artery disease and a cardiac disease that stimulates enlargement of the epicardial artery with a bioresorbable scaffold in a patient in need thereof comprising: implanting the bioresorbable stent at stenotic lesion in a segment of an epicardial artery of the patient which increases a diameter of the stenotic segment of the artery, wherein the patient has a cardiac disease that stimulates enlargement of the epicardial artery resulting from an increase in basal blood flow, wherein the bioresorbable stent is configured to support the segment at the increased diameter to treat the stenosis and restore freedom of radial movement of the segment through a decrease in radial strength, loss of mechanical integrity, and resorption of the bioresorbable stent, and wherein the restoration of freedom of radial movement of the segment provides expansive remodeling of the segment required by the increase in basal blood flow.

The cardiac disease may be selected from the group consisting of dilated cardiomyopathy, hypertrophic cardiomyopathy, aortic valve disease, mitral valve regurgitation, thyroid disease, left ventricular hypertrophy, anemia, or any other disease that increases the demand for blood flow.

The cardiac disease may be left ventricular hypertrophy.

The cardiac disease may be dilated cardiomyopathy, wherein the patient has an enlarged heart cavity. The patient may have Barth syndrome which caused the dilated cardiomyopathy. The patient may be a male child under the age of 14 years. The patient may be a pregnant woman or a woman that developed the dilated cardiomyopathy during pregnancy or after childbirth.

The cardiac disease may be hypertrophic cardiomyopathy and a muscle mass of the patient's left ventricle is enlarged. The hypertrophic cardiomyopathy may be hypertrophic obstructive cardiomyopathy, a septum between a left and right heart ventricles of the patient is enlarged and obstructs blood flow from the left ventricle. The enlarged septum may distort one leaflet of a mitral valve of the patient causing leakage from the mitral valve. The patient may be a child between 12 and 18 years old.

The cardiac disease may be aortic valve disease. The aortic valve disease may be aortic regurgitation, wherein the patient's aortic valve is widened or weakened which causes blood to flow backward from into the patient's left ventricle. The patient may have holes in leaflets of the aortic valve which causes the aortic regurgitation. The aortic regurgitation may be caused by a condition selected from the group consisting of rheumatic fever, a bicuspid aortic valve, a high blood pressure including a diastolic pressure more than 110 mm Hg, Marfan syndrome, endocarditis, ankylosing spondylitis, and dissecting aortic aneurysm. The aortic valve disease may comprise aortic stenosis comprising a narrowing or blockage of the aortic valve of the patient. The leaflets of the aortic valve may be coated with deposits that alter a shape of the leaflets and reduce blood flow through the valve.

The cardiac disease may be mitral valve regurgitation in which the mitral valve of the patient allows leaking of blood backward into the patient's heart due to damage to the valve that prevents tight closure of the valve. The mitral valve regurgitation may be chronic. The chronic mitral valve regurgitation may be caused by a condition selected from the group consisting of heart failure, rheumatic fever, congenital heart disease, and calcium buildup in the valve. The mitral valve regurgitation may be acute. The acute mitral valve regurgitation may be due to rupture of the mitral valve or nearby tissue. The acute mitral valve regurgitation may be due to heart attack or endocarditis.

The cardiac disease may be hyperthyroidism. The cardiac disease may be anemia.

A second embodiment of the present invention may include a method of treating coronary artery disease) and an event that precipitates enlargement of an epicardial artery with a bioresorbable scaffold in a patient in need thereof comprising: implanting the bioresorbable stent at a stenotic lesion in a segment of the epicardial artery of the patient, wherein the patient has experienced an event that precipitates enlargement of the epicardial artery resulting from an increase in basal blood flow, wherein the bioresorbable stent is configured to support the segment at the increased diameter to treat the stenosis and restore freedom of radial movement of the segment through a decrease in radial strength, loss of mechanical integrity, and resorption of the bioresorbable stent, and wherein the restoration of freedom of radial movement of the segment provides expansive remodeling of the segment required by the increases in basal blood flow. The event may be a myocardial infarction. The event may be damage to a mitral valve.

A third embodiment of the present invention includes a method of treating coronary artery disease in a patient in need thereof that is engaging in an endurance exercise program that stimulates enlargement of the epicardial artery with a bioresorbable scaffold comprising: implanting the bioresorbable stent at stenotic lesion in a segment of an epicardial artery of the patient which increases a diameter of the stenotic segment of the artery, wherein the patient is engaging in an exercise program that stimulates enlargement of the epicardial artery resulting from an increase in basal blood flow, wherein the bioresorbable stent is configured to support the segment at the increased diameter to treat the stenosis and restore freedom of radial movement of the segment through a decrease in radial strength, loss of mechanical integrity, and resorption of the bioresorbable stent, and wherein the restoration of freedom of radial movement of the segment provides expansive remodeling of the segment required by the increase in basal blood flow.

The treatment of the cardiac disease that stimulates enlargement of epicardial arteries, an event that precipitates enlargement of epicardial arteries, or in a patient engaging in an endurance exercise program that stimulates enlargement of epicardial arteries may further include the following. The scaffold may cause lumen area of the segment to increase from implantation to 42 months post-implantation. The scaffold may cause an area circumscribed by an external elastic lamina (EEL) of the segment to increase from 1 month to 30 months post-implantation. The scaffold may cause the EEL area to stabilize at or about 30 months post-implantation and be stable 30 to 40 months or beyond 40 months post-implantation. Stable refers to no trend upward or downward during a specified time period. The scaffold may cause a neointimal area of the segment to increase between implantation and 1 month post-implantation. The scaffold may cause the neointimal area to be stable 1 to 20 months or beyond 20 months post-implantation. The scaffold may cause a medial area of the segment to be stable throughout post-implantation of the scaffold. The scaffold may cause a wall thickness of the artery to increase between implantation and 3 months post-implantation. The scaffold may cause the wall thickness to be approximately constant after 3 months post-implantation. The deployment of the scaffold increases circumferential wall stress (CWS) on the segment. The scaffold causes decrease of the CWS between 1 and 3 months post-implantation. The scaffold decreases the CWS between 3 and 20 months post-implantation and beyond 20 months post-implantation. The scaffold may cause the wall shear stress (WSS) to increase between deployment and 3 months post-implantation. The scaffold may cause the WSS to decrease between 3 months and 20 months post-implantation and decrease beyond 20 months post-implantation.

### BRIEF DESCRIPTION OF THE DRAWINGS

FIG. 1 depicts an exemplary stent scaffold.

FIG. 2A depicts a bioresorbable vascular scaffold (BVS) in a crimped configuration.

FIG. 2B show a cross-selection of a strut of the BVS of FIG. 2A.

FIG. 3 depicts changes in OCT lumen area as a function of time for BVS- and Xience-treated porcine coronary arteries.

FIG. 4A depicts area circumscribed by the External Elastic Lamina (EEL) changes as a function of time for BVS-treated and Xience-treated porcine coronary arteries.

FIG. 4B depicts neointimal area changes as a function of time for BVS-treated and Xience-treated porcine coronary arteries.

FIG. 4C depicts medial area changes as a function of time for BVS-treated and Xience-treated porcine coronary arteries.

FIG. 4D depicts wall thickness changes as a function of time for BVS-treated and Xience-treated porcine coronary arteries

FIG. 5A depicts circumferential wall stress changes as a function of time for BVS- and Xience-treated porcine coronary arteries.

FIG. 5B depicts wall shear stress changes as a function of time for BVS- and Xience-treated porcine coronary arteries.

FIG. 6A depicts lumen area effects of BVS or Xience placement on responses of porcine coronary arteries to increases in body weight.

FIG. 6B depicts neointimal area effects of BVS or Xience placement on responses of porcine coronary arteries to increases in body weight.

FIG. 6C depicts EEL area effects of BVS or Xience placement on responses of porcine coronary arteries to increases in body weight.

FIG. 7A depicts a radial cross-section of an artery.

FIG. 7B depicts a section of an artery in three dimensions.

### INCORPORATION BY REFERENCE

All publications and patent applications mentioned in this specification are herein incorporated by reference to the same extent as if each individual publication or patent application was specifically and individually indicated to be incorporated by reference, and as if each said individual publication or patent application was fully set forth, including any figures, herein.

### DETAILED DESCRIPTION OF THE INVENTION

Various embodiments of the present invention include treatment of patients that have coronary artery disease (CAD) with a bioresorbable scaffold that have diseases that cause epicardial arteries to enlarge or treatment of patients that have epicardial arteries that enlarge due to exercise. Additionally, embodiments include treatment of patients with a bioresorbable scaffold of patients who have experienced events that could precipitate one or more of the diseases. Also, embodiments include treatment of patients with a bioresorbable scaffold of patients who are engaged in an endurance exercise program that stimulates enlargement of epicardial arteries.

The bioresorbable stent can include a support structure in the form of a scaffold made of a material that is bioresorbable, for example, a bioresorbable polymer such as a lactide-based polymer. A bioabsorbable, bioresorbable, bioerodible stent may also have a bioerodible metallic body. The scaffold is designed to completely erode away from an implant site after treatment of an artery is completed. The scaffold can further include a drug, such as an antiproliferative or anti-inflammatory agent. A polymer coating disposed over the scaffold can include the drug which is released from the coating after implantation of the stent. The polymer of the coating may also be bioresorbable.

A stent scaffold is composed of a plurality of structural elements having a tubular shape. Such scaffolds can include a plurality of cylindrical rings connected or coupled with linking elements. For example, the rings may have an undulating sinusoidal structure. When deployed in a section of a vessel, the cylindrical rings are load bearing and support the vessel wall at an expanded diameter or a diameter range due to cyclical forces in the vessel. Load bearing refers to the supporting of the load imposed by radial inwardly directed forces. Structural elements, such as the linking elements or struts, are generally non-load bearing, serving to maintain connectivity between the rings. For example, a stent may include a scaffold composed of a pattern or network of interconnecting structural elements or struts.

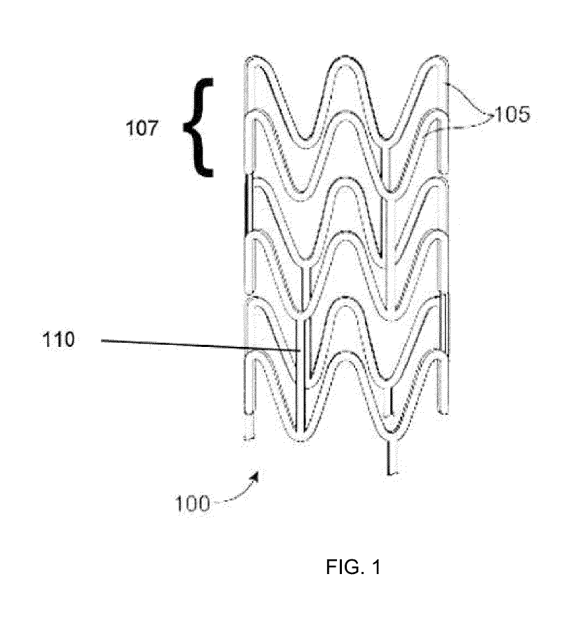

FIG. 1 depicts a view of an exemplary stent 100. In some embodiments, a stent may include a body, backbone, or scaffold having a pattern or network of interconnecting structural elements 105. Stent 100 may be formed from a tube (not shown). FIG. 1 illustrates features that are typical to many stent patterns including undulating sinusoidal cylindrical rings 107 connected by linking elements 110. The cylindrical rings are load bearing in that they provide radially directed force to support the walls of a vessel. The linking elements generally function to hold the cylindrical rings together. A structure such as stent 100 having a plurality of structural elements may be referred to as a stent scaffold or scaffold. Although the scaffold may further include a coating, it is the scaffold structure that is the load bearing structure that is responsible for supporting lumen walls once the scaffold is expanded in a lumen.

The structural pattern in FIG. 1 is merely exemplary and serves to illustrate the basic structure and features of a stent pattern. A stent such as stent 100 may be fabricated from a polymeric tube or a sheet by rolling and bonding the sheet to form the tube. A tube or sheet can be formed by extrusion or injection molding. A stent pattern, such as the one pictured in FIG. 1, can be formed on a tube or sheet with a technique such as laser cutting or chemical etching. The stent can then be crimped on to a balloon or catheter for delivery into a bodily lumen. In other embodiments, the scaffold design could be braided polymer filaments or fibers.

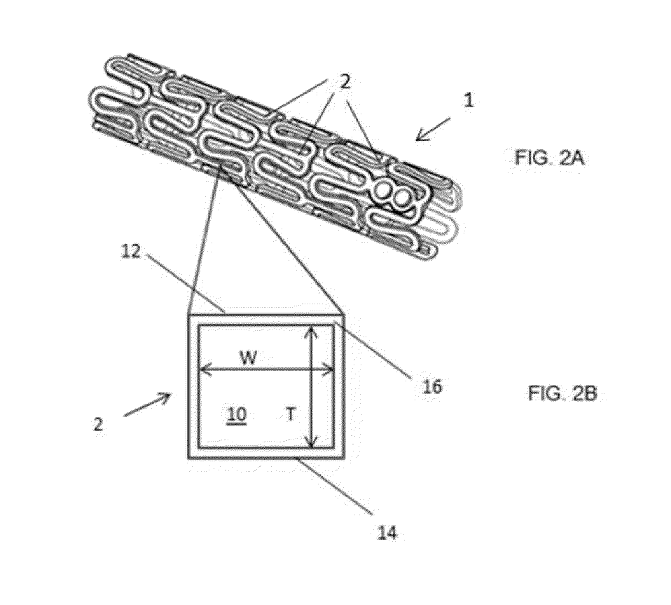

FIG. 2A depicts a bioresorbable vascular scaffold (BVS) 1 composed of a plurality of struts 2 in a crimped configuration. FIG. 2B shows a cross-selection of a strut 2 showing the polymer scaffold body, polymer backbone, or core of the strut surrounded by a drug/polymer coating or matrix 16. The cross-section of the strut has an abluminal surface or side 12 that faces the vessel wall and a luminal surface or side 14 that faces the lumen of the vessel. The strut cross-section shown is rectangular with a width (W) and thickness (T). The scaffold cross-section may be approximately square with an aspect ratio T/W close to 1.

Coronary artery disease is a narrowing or blockage of the arteries and vessels that provide oxygen and nutrients to the heart. It is caused by atherosclerosis, an accumulation of fatty materials on the inner linings of arteries. The accumulation results in the formation of a stenosis or stenotic lesion. The resulting stenosis restricts blood flow to the heart. When the blood flow is completely cut off, the result is a heart attack.

Coronary artery disease occurs when the coronary arteries become partially blocked or clogged. This blockage limits the flow of blood from the coronary arteries, which are the major arteries supplying oxygen-rich blood to the heart. The coronary arteries expand when the heart is working harder and needs more oxygen. If the arteries are unable to expand, the heart is deprived of oxygen (myocardial ischemia). When the blockage is limited, chest pain or pressure, called angina, may occur. When the blockage cuts off the flow of blood, the result is heart attack (myocardial infarction or heart muscle death).

There are a number of cardiac diseases that stimulate epicardial coronary artery diameter increases. The diseases include those that increase cardiac mass and metabolism as a result of increase in basal blood flow. The diseases include, but are not limited to, dilated cardiomyopathy, hypertrophic cardiomyopathy, aortic valve disease, mitral valve regurgitation, thyroid disease, left ventricular hypertrophy, and anemia. Events that could precipitate such diseases include myocardial infarction or damage to a mitral valve or aortic valve.

Epicardial artery enlargement in patients with these diseases has been documented by several investigators. Roberts et al. reported in Circulation 62: 953-959, 1980 that the area circumscribed by the internal elastic lamina (IEL), used to estimate the lumen, was greater in hearts obtained from patients with healed myocardial infarcts with progressive and eventually fatal congestive heart failure (8.6 mm²) and patients with aortic valve disease (9.6 mm²) than from patients with angina (6.0 mm²). Importantly, IEL areas were directly related to heart weights which were 588 gm, 730 gm, and 386 gm, respectively. Villari et al. reported in J Am Coll Cardiol 20: 1073-1079, 1992 that coronary artery cross-sectional lumen areas were increased in patients with aortic valve disease over 66 months follow-up and that the increases were directly related to left ventricular muscle mass increases (10.3-10.9 mm²/100 g). More recently, Windecker et al. reported in Am J Physiol 282: H2216-H2223, 2002 that 5 months of endurance training of healthy volunteers increased angiographic cross-sectional area of the left main, left anterior descending, and right coronary artery in parallel with increases in left ventricular mass index (gm/m²).

Table 1 shows effects of cardiac diseases and physiologic stresses on epicardial coronary artery diameter and flow reserve. Epicardial flow reserve (EFR) is defined as the ratio of maximum flow in the presence of a disease to normal maximum flow (control). The EFR is a site-specific index of artery enlargement that can be calculated by simultaneous measurement of mean arterial, distal coronary, and central venous pressure (Pa, Pd, and Pv, respectively), during pharmacological vasodilation.

TABLE 1
Effects of cardiac diseases and physiologic stresses on epicardial coronary artery diameter and flow reserve.

| | | | | | EPICARDIAL |
|---|---|---|---|---|---|
| AUTHOR | DISEASE | ARTERY | | | FLOW RESERVE |
| | | | DIAMETER | DIAMETER | |
| | | | (CONTROL) | (DISEASED) | |
| Dodge | LVH | LAD | 3.6 | 3.9 | 1.38 |
| | | LCx | 3.4 | 3.6 | 1.26 |
| | | RCA | 3.9 | 4.6 | 1.94 |
| | dilated cardiomyopathy | LAD | 3.6 | 3.8 | 1.24 |
| | | LCx | 3.4 | 3.3 | 0.89 |
| | | RCA | 3.9 | 4.5 | 1.77 |
| Kimbell | aortic valve stenosis | LAD | 3.32 | 3.82 | 1.75 |
| | hypertrophic cardiomyopathy | LAD | 3.32 | 4.72 | 4.09 |
| | | | CROSS-SECTIONAL | CROSS-SECTIONAL | |
| | | | AREA (CONTROL) | AREA (DISEASED) | |
| Kaufman | hypertrophic cardiomyopathy | LAD | 7.8 | 13.5 | 3.00 |
| | | LCx | 6.7 | 11.6 | 3.00 |
| | | RCA | 9 | 12.2 | 1.84 |
| | dilated cariomyopathy | LAD | 7.8 | 12 | 2.37 |
| | | LCx | 6.7 | 10.6 | 2.50 |
| | | RCA | 9 | 14.3 | 2.52 |
| Roberts | angine | coronary mean area | 5.5 | 6 | 0.85 |
| | healed MI + fatal CHF | coronary mean area | 6.5 | 8.6 | 1.75 |
| | MI | coronary mean area | 6.5 | 7.6 | 1.37 |
| | sudden cardaic death | coronary mean area | 6.5 | 7.6 | 1.37 |
| | healed MI + noncardiacdeath | coronary mean area | 6.5 | 6.9 | 1.13 |
| | aortic valve | coronary mean area | 6.5 | 9.6 | 2.18 |
| Villari | aortic valve disease | LAD | 8 | 13 | 2.64 |
| | aortic valve 66 mo FU | LAD | 8 | 17 | 4.52 |
| | aortic valve disease | LCx | 6 | 13 | 4.69 |
| | aortic valve 66 mo FU | LCx | 6 | 15 | 6.25 |
| Windecker | 5 mo endurance exercise | LM | 16.1 | 17.1 | 1.13 |
| | | LAD | 9.5 | 10.1 | 1.13 |
| | | LCx | 5 | 5.2 | 1.08 |
| | | RCA | 10.1 | 15.1 | 2.24 |
| Kozakova 2007 | athletes physiologic LVH | left main | 13.2 | 17.5 | 1.76 |
| | untreated HT with no LVH | left main | 13.2 | 10.1 | 0.59 |
| | untreated HT with LVH | left main | 13.2 | 13.1 | 0.98 |

Flow = pressure difference × π radius⁴/8 nl
Assume all constant except radius⁴
Area = π × radius²
MI—myocardial infarction
FU—follow-up
Flow reserve = (treatment radius⁴/control radius⁴)
Dodge JT, et al. 1992; 86: 232-246.
Kimball BP, et al. Am J Cardiol 1990; 65: 767-771.
Kaufmann P, et al. JACC 1996; 745-750.
Kozakova M, et al. Am J Hypertens 2007; 20: 279-284.

Overall, these results suggest that as the heart increases in muscle mass, basal epicardial coronary artery blood flow, and therefore wall shear stress (WSS), increase. In response, the coronary arteries undergo expansive remodeling and increase epicardial flow potential.

In general, arteries exhibit control mechanisms that provide “tensional homeostasis”. These mechanisms act to maintain circumferential wall stress (CWS), wall shear stress (WSS), and axial wall stress (AWS) at constant levels. For example, large conducting arteries maintain stresses near values of 1.5 Pa for WSS and 100 kPa for AWS and WSS. [Humprhrey, J D Hypertension 52: 195-200, 2008] The process of performing balloon angioplasty disrupts these control mechanisms by increasing CWS and decreasing WSS. [Consigny P M, ATVB 1986; 6: 265-276; Wentzel J J, et al. Circulation 103: 1470-1745, 2001]. The artery responses include intimal thickening and vascular remodeling (restrictive or expansive) that bring CWS and WSS back towards normal (Wentzel 2001). However, these responses lead to lumen loss or restenosis. [Post M J, et al. Circulation 89: 2816-2821, 1994; Mintz G S, et al. Circulation 1996; 94: 35-43; De Smet B J G L, et al. Cardiovasc Res 1998; 38: 224-232]

BMS were introduced to eliminate problems associated with balloon angioplasty including acute elastic recoil and dissections and long-term restrictive remodeling. Subsequently, DES were introduced to decrease the greater intimal thickening elicited by the vascular wall stresses and injury associated with stent placement. [Timmins L H, et al. 2011; 91: 955-967]

If a metallic stent, such as a DES or BMS, were placed in a patient, the artery would not be able to expand to meet an increase blood flow demand any time after implantation necessitated by the above-mentioned diseases. An inability to meet such demand can lead to ischemic complications such as angina, myocardial hibernation, or myocardial infarction.

The fact that the BVS is bioresorbable suggests that the homeostatic responses of the artery to BVS placement should be different from the response to durable metallic stent placement. However, a comparison has yet to be published that characterizes CWS and WSS changes in arteries treated with durable metallic stents and BVS. In particular, there is no study that compares the responses of arteries susceptible to enlarging treated with a DES and BVS.

A study was performed to evaluate and compare the effect of deployment of a metallic DES (Xience, Abbott Vascular, Santa Clara, Calif.) vs. a BVS, (ABSORB, Abbott Vascular, Santa Clara, Calif.) on arterial homeostasis. DES and BVS were implanted in the epicardial coronary arteries of 71 Yucatan mini-swine. The arteries were evaluated using angiography and optical coherence tomography (OCT) at the time of deployment and at the time of vessel harvesting 1, 3, 6, 12, 18, 24, 30, 36, or 42 months later.

Histomorphometric measurements were used to estimate CWS; OCT-derived lumen area and blood flow, derived from body weight, were used to estimate WSS.

DES- and BVS-treated arteries responded to changes in CWS and WSS differently. In response to device deployment and increase in CWS, DES-treated arteries responded through neointima formation and BVS-treated arteries responded through formation of neointima and medial thickening. CWS was significantly greater in the Xience-treated arteries presumably due to shielding by the stent.

After device deployment, increases in body weight increased WSS. DES-treated arteries responded through intimal thinning whereas BVS-treated arteries responded through expansive remodeling resulting in a greater maximal flow conductance.

The results suggest that, in the long term, BVS-treated arteries have a greater flow reserve which should be beneficial in conditions where flow demand is increased such as the cardiac diseases and conditions disclosed herein. The study further suggests that a BVS deployment does not inhibit epicardial coronary artery diameter increases. Therefore, a BVS may provide a unique advantage to these patients having the above-mentioned cardiac diseases and conditions. As result, the patients have greater flow reserves and fewer ischemic complications.

The interaction of the BVS and DES with an artery can be characterized in three phases: a revascularization phase, restoration phase, and a remodeling phase.

In the revascularization phase, both BVS and DES serve as a scaffold to support an increased arterial diameter. For both devices, the lumen diameter is reduced due to medial thickening and neotintima formation. The revascularization phase lasts from implantation to 3 to 4 months post-implantation.

In the restoration phase, the DES maintains its structural rigidity and the BVS begins to lose its radial strength. In a DES-treated artery, the EEL area remains unchanged, indicating no remodeling occurs.

In BVS-treated arteries, the EEL expands over time demonstrating that these arteries can undergo the expansive remodeling that is needed to adjust for increases in basal coronary flow increases. The restoration phase may start at a time after implantation greater than 3 months, after the minimum time that vessel support is required, or when the radial strength falls below a selected value, such as 500 mmHg, 300 mmHg, or 200 mmHg. In the remodeling phase, CWS and WSS stabilize in both the DES and BVS-treated arteries. WSS stabilizes by expansive remodeling in the BVS-treated arteries and by neointimal remodeling and thinning in the DES-treated arteries.

The above-mentioned patients with these diseases or conditions may have stenotic lesions in a segment of an epicardial artery. A method of treatment includes positioning a BVS at the lesion or stenotic segment of the artery and expanding the scaffold at the segment which increases the diameter of the segment. The scaffold treats the stenosis by maintaining patency of the segment for a period of time sufficient for the segment to maintain an increased diameter in absence of the scaffold.

The radial strength of the scaffold eventually decreases after this period of time due to degradation of the scaffold polymer. In addition, a neointima layer forms over the scaffold between 1 and 6 months after implantation.

After the radial strength decreases, the scaffold begins to lose mechanical or structural integrity which includes by struts of the scaffold breaking up. The scaffold erodes or absorbs from the vessel leaving a healed vessel segment. The decrease in radial strength, breaking up of the scaffold, and erosion of the scaffold restores freedom of radial movement to the segment.

During and after restoration of the radial movement, the epicardial artery may expand at the implant site in response to increasing basal flow-induced remodeling necessitated by the above-mentioned diseases or conditions.

When a durable metallic stent, such as a BMS or DES, is implanted into an epicardial artery susceptible to or in the process of expansive remodeling, expansive remodeling cannot occur after deployment of the BMS or DES. However, after implantation of a BVS in such an epicardial artery, expansive remodeling or further expansive remodeling can occur. Specifically, expansive remodeling of an epicardial artery treated with a BVS can occur once the radial strength decreases and the scaffold loses mechanical integrity sufficiently to allow outward radial movement of the artery.

For both a BVS and a durable a metallic stent, a layer of neointima develops over the implant between the implant and the blood flow in the vessel lumen. In an artery treated with a durable metallic stent, remodeling may include intimal thinning to increase the flow potential necessitated by increasing basal flow-induced remodeling. However, such thinning could result in exposure of the underlying metallic stent to blood flow. Such exposure could lead to thrombosis and complications resulting from the thrombosis.

In some embodiments, a BVS is implanted in the artery of patient prior to an increase in basal flow or prior to epicardial artery enlargement. Basal flow increase may occur after implantation and before complete resorption of the BVS. Epicardial enlargement may also occur after implantation and before complete resorption of the BVS. In other embodiments, there is no basal flow increase or epicardial enlargement between implantation and complete resorption of the BVS.

In some embodiments, a BVS is implanted in the artery of patient after an event that can stimulate epicardial coronary artery enlargement. The event may include myocardial infarction, damage to a mitral valve, or damage to an aortic valve. The implantation may be within 1 day after the event, 1 to 5 days after the event, 5 to 30 days after the event, 30 to 60 days after the event, or more than 60 days after the event.

In some embodiments, a method of treatment includes treating CAD in a patient prior to or during engagement of the patient in an endurance exercise program. An endurance exercise program may refer to a program in which the patient engages an activity greater than 3 to 7 hours per week or greater than 7 hours per week of exercise activity with a heart rate at least 65% of the patient's maximum heart rate (MHR), or more narrowly, 65 to 80% of the patient's MHR.

The MHR is defined as the highest heart rate in beats per minute that can be attained by an individual in strenuous activity, varying with fitness and, in adults, inversely with age. A “rule-of-thumb” formula for the MHR is 220 minus age in years. The most accurate measurement is via a cardiac stress test. In this test, a person is subjected to controlled physiologic stress (generally by treadmill) while being monitored by an ECG. The intensity of exercise is periodically increased until certain changes in heart function are detected on the ECG monitor, at which point the subject is directed to stop. Typical duration of the test ranges ten to twenty minutes. Various formulas are employed to estimate the MHR, for example, in Tanaka H, et al. J. Am. Coll. Cardiol, 37 (1): 153-6; Gellish R L, et al., Med Sci Sports Exerc 39 (5): 822-9; Robergs R et al. Journal of Exercise Physiology 5 (2): 1-10; Galati M, et al. Circulation 122 (2): 130-7.

The EFR provided by any of the treatments with a BVS disclosed herein can be 1.1 to 6, 1.1 to 1.5, 1.5 to 2, 2 to 2.5, 2.5 to 3, 2 to 3, 2 to 4, 3 to 4, 4 to 5, 5 to 6, or greater than 6, The EFR may be determined 1 to 2 years, 2 to 3 years, 3 to 4 years, 4 to 5 years, or greater than 5 years after implantation of the BVS.

The method of treatment includes treatment of a patient with a BVS having CAD and a cardiac disease that stimulate epicardial coronary artery diameter increases, the cardiac disease includes dilated cardiomyopathy, hypertrophic cardiomyopathy, aortic valve disease, mitral valve regurgitation, thyroid disease, left ventricular hypertrophy, or anemia. A patient treated may have any of the symptoms described below associated with the cardiac disease.

Left ventricular hypertrophy (LVH) is enlargement (hypertrophy) of the muscle tissue that makes up the wall of your heart's main pumping chamber (left ventricle). Left ventricular hypertrophy develops in response to some factor, such as high blood pressure, that requires the left ventricle to work harder. As the workload increases, the walls of the chamber grow thicker, lose elasticity and eventually may fail to pump with as much force as that of a healthy heart.

Cardiomyopathy is a chronic disease of the heart muscle (myocardium), in which the muscle is abnormally enlarged, thickened, and/or stiffened. The weakened heart muscle loses the ability to pump blood effectively, resulting in irregular heartbeats (arrhythmias) and possibly even heart failure. Cardiomyopathy primarily affects the left ventricle, which is the main pumping chamber of the heart. The disease is often associated with inadequate heart pumping and other heart function abnormalities.

With dilated or congestive cardiomyopathy, the heart cavity is enlarged and stretched (cardiac dilation), which results in weak and slow pumping of the blood, which in turn can result in the formation of blood clots. A genetically-linked cardiac disease, Barth syndrome causes dilated cardiomyopathy. This syndrome affects male children, and is usually diagnosed at birth or within the first few months of life. Pregnant women during the last trimester of pregnancy or after childbirth may develop a type of dilated cardiomyopathy referred to as peripartum cardiomyopathy.

With hypertrophic cardiomyopathy, the muscle mass of the left ventricle enlarges, or hypertrophies. In hypertrophic obstructive cardiomyopathy (HOCM), the septum (wall) between the two heart ventricles (the pumping chambers) becomes enlarged and obstructs blood flow from the left ventricle. The thickened wall can also distort one leaflet of the mitral valve, which results in leakage. HOCM is most common in young adults and is often hereditary, caused by genetic mutations in the affected person's DNA.

The aortic valve regulates the blood flow from the heart's lower-left chamber (the left ventricle) into the aorta. The aorta is the main vessel that supplies blood to the rest of the body. The most common and serious valve problems happen in the mitral and aortic valves. Aortic regurgitation and aortic stenosis are two common aortic valve diseases.

Aortic regurgitation is also called aortic insufficiency or aortic incompetence. It is a condition in which blood flows backward from a widened or weakened aortic valve into the heart's lower chamber (the left ventricle). The most serious form of aortic regurgitation is caused by an infection that leaves holes in the valve leaflets. Symptoms of aortic regurgitation may not appear for years. When symptoms do appear, it is because the left ventricle must work harder to make up for the backflow of blood. The ventricle eventually gets larger, causing a backup of fluid.

The most common cause of severe aortic regurgitation is rheumatic fever. Mild cases are often caused by a bicuspid aortic valve (where the valve has 2 leaflets instead of 3) and severe high blood pressure (a diastolic pressure more than 110 mm Hg). Other causes, though rare, may include Marfan syndrome, Endocarditis, Ankylosing spondylitis (arthritis of the spine), and dissecting aortic aneurysm.

Symptoms begin because the left ventricle has to work harder. In time, the ventricle gets larger and fluid backs up. Symptoms may include shortness of breath, chest pain that gets worse with exercise and goes away with rest, swelling in the ankles, fatigue, fast or fluttering pulse, severe cases can lead to heart failure. In most of these cases, the aortic valve will eventually need to be replaced to fix the backflow of blood into the left ventricle.

Aortic stenosis is a narrowing or blockage of the aortic valve. This valve regulates the blood flow from the heart's lower-left chamber (the left ventricle) into the aorta. The aorta is the main blood supplier to the rest of the body. Aortic stenosis happens when the valve leaflets become coated with deposits. The deposits change the shape of the leaflets and reduce blood flow through the valve. The left ventricle has to work harder to make up for the reduced blood flow. Over time, the extra work can weaken the heart muscle.

The mitral valve, also known as the bicuspid valve or left atrioventricular valve, is a dual-flap valve in the heart that lies between the left atrium (LA) and the left ventricle (LV). Mitral valve regurgitation refers to a condition in which the mitral valve is letting blood leak backward into the heart. The mitral valve is on the left side of your heart and lets blood flow from the upper to the lower heart chamber. Mitral valve regurgitation occurs when it is damaged and may no longer close tightly which lets blood leak backward, or regurgitate, into the upper chamber. As a result, the heart has to work harder to pump this extra blood. There are two forms of mitral valve regurgitation: chronic and acute.

Chronic mitral valve regurgitation may develop slowly due to wear and tear as a person gets older. Other causes include heart failure, rheumatic fever, congenital heart disease, a calcium buildup in the valve, and other heart problems.

Acute mitral valve regurgitation develops quickly and happens when the valve or nearby tissue ruptures suddenly. Instead of a slow leak, blood builds up quickly in the left side of the heart. Common causes of acute regurgitation are heart attack and a heart infection called endocarditis.

The thyroid gland is a small gland located in the neck that secretes hormones responsible for regulating many vital bodily functions including the heart. Disorders of the thyroid gland generally cause one of two types of problems: production of too little thyroid hormone (hypothyroidism) or production of too much thyroid hormone (hyperthyroidism).

Hyperthyroidism is caused by the overproduction of thyroid hormone. Excess thyroid hormone increases the force of contraction of the heart muscle, and increases the amount of oxygen demanded by the heart. It also increases the heart rate and, as a result, the work of the heart is greatly increased in hyperthyroidism.

Anemia is a below-normal level of hemoglobin or hematocrit. Hemoglobin is the protein in red blood cells that carries oxygen to all parts of the body. Anemia can be a temporary condition, a consequence of other health conditions, or it can be a chronic problem.

Anemia can lead to severe chest pain because parts of the heart are not getting enough oxygen. Lack of oxygen makes a heart work harder, so the muscles in its left-lower chamber may get too thick. This may result in an enlarged heart and epicardial arteries.

Endurance exercise increases the demand for oxygen which makes the heart work harder. A study has shown that a long term endurance exercise program (5 months) results in epicardial coronary artery size and vasodilatation increase. In addition, the capacity to augment coronary flow during hyperemia improves in response to a sustained endurance exercise program.

### Example

An animal study was performed which included implanting two types of devices, metallic and polymeric. The metallic devices were cobalt chromium, balloon-expandable stents coated with a fluorinated copolymer containing everolimus (100 μg/cm²) (Xience, Abbott Vascular, Santa Clara, Calif.). The polymeric devices were bioresorbable, balloon-expandable vascular scaffolds (BVS) with a poly(L-lactide) (PLLA) backbone and a poly(D,L-Lactide) PDLLA coating containing everolimus (100 μg/cm²) (ABSORB BVS, Abbott Vascular, Santa Clara, Calif.). The BVS investigated in this study is the same construct as that used in Cohort B of the ABSORB clinical trial. [Serruys P W, et al. Circulation 2010; 122: 2301-2312]

71 Yucatan mini-swine were studied using a protocol that was approved by the institution's Animal Care and Use Committee. For each pig, a single Xience or BVS was randomly implanted in each of the 3 main coronary arteries in each pig using an implant ratio of 2 BVS to 1 Xience. Prior to implantation, each pig was weighed and then sedated with an intramuscular injection of ketamine (0.04 mg/kg, azaperone (4.0 mg/kg) and atropine (25 mg/kg). Anesthesia was induced by an intravenous injection of propofol (1.66 mg/kg) and maintained with the inhalation of isofluorane (1-3%). A vascular access sheath was then placed in a femoral artery and heparin (5000-10000 U) was injected into the artery to maintain an active clotting time greater than 250 seconds. A guiding catheter was then advanced retrogradely and positioned in the left main and right coronary arteries and, after the intracoronary injection of nitroglycerine, angiograms were recorded. The angiographic images were used to select a segment of coronary artery where device deployment could be achieved using a balloon-to-artery ratio of 1.1:1. The selected device was then deployed by inflating the balloon to the appropriate pressure selected from the balloon pressure-volume (compliance) curve. After all devices had been implanted, follow-up angiographic images were captured. Thereafter, OCT imaging was performed (M2, LightLab Imaging or C7-XR, St. Jude Medical). An OCT catheter was advanced through the guiding catheter into the distal coronary artery and the coronary artery and implanted device were imaged with motorized pullback (M2: 1 mm/sec and C7: 20 mm/sec) during the infusion of contrast media (2-4 ml/min) through the guiding catheter.

The implanted devices were removed 1 (n=7 pigs), 3 (n=9 pigs), 6 (n=9 pigs), 12 (n=8 pigs), 18 (n=7 pigs), 24 (n=7 pigs), 30 (n=8 pigs), 36 (n=8 pigs), or 42 (n=8 pigs) months after implantation. Each animal received 1 or 2 BVS (3.0×18 mm at 1, 3, and 6 months and 3.0×12 mm at 12, 18, 24, 30, 36, and 42 months) and 1 comparable length EES in the main coronary arteries. As described above, each pig was weighed, sedated, and anesthetized. A vascular sheath was placed in a femoral artery, heparin was injected, and coronary angiography and OCT were performed. Each pig was then euthanized and the heart was removed and pressure perfused with 0.9% saline.

Following saline perfusion, each heart was perfusion fixed with 10% buffered formalin at an intraluminal pressure of 80-100 mmHg. After fixation, the coronary arteries were dissected, cut into pieces representing the proximal, middle and distal portions of each device and the artery proximal and distal to the device. The pieces of artery were dehydrated in graded series of ethanol solutions, embedded in methyacrylate, sectioned (4-6 um thick) and stained with Movat's pentachrome stain.

Histomorphometry was performed by computerized planimetry using Image-Pro Plus software (MediaCybernetics, Rockville, Md.) on the three representative sections. For each section, the circumferences of the lumen, internal elastic lamina (IEL), and external elastic lamina (EEL) were traced. Neointimal area was calculated as the IEL area minus lumen area. Medial area was calculated as EEL area minus IEL area. Wall thickness was calculated as the diameter of the EEL minus the diameter of the lumen assuming that the EEL and lumen were circular.

Quantitative coronary angiographic images were obtained prior to device implantation, at the time of balloon inflation, shortly after device implantation and at the time of explant. Angiograms (Siemens AXIOM—Ards, Munich, Germany) were acquired and analyzed using a PC-based quantitative coronary angiography (QCA) workstation (Siemens Leonardo, Munich, Germany). Average lumen diameters of the treated segment were measured using the automated edge detection algorithm of the system with the guiding catheter as the reference for calibration.

From the OCT pullback, three representative still frames from the proximal, mid and distal regions of the implanted segment were captured and lumen areas measured using onboard software with z-offset calibrated to the imaging catheter.

CWS and WSS were approximated using the following methods. (Humphrey J D Hypertension 52: 195-200, 2008) WSS was calculated as (4×blood viscosity×volumetric flow)/(π×luminal radius³). For this calculation, whole blood viscosity for the pig was assumed to be 0.05 gm/cm/sec (determined at a shear rate of 94 s⁻¹ by Windberger U et al., Experimental Physiology 2003; 88: 431-440. Total volumetric blood flow to the coronary arteries was assumed to be equal to 5% of cardiac output at rest; cardiac output was assumed to be equal to (body mass)^{0.75} [Holt J P, et al. Am J Physiol 215: 704-715, 1968] [West, G B, et al. Science 276: 122-126, 1997] [Weinberg P D et al. J Biomechanics 40: 1594-1598, 2007]. Volumetric flow to each coronary artery was assumed to be one-third of total coronary blood flow. Luminal radius was calculated from the OCT measured lumen area. CWS was calculated according to the Law of Laplace as (arterial pressure×luminal radius)/wall thickness). [Humphrey 2008] Arterial pressure was assumed to be 100 mmHg for all pigs. Luminal radius and wall thickness were derived from histomorphometric measurements.

All statistical analyses were performed using JMP (Version 10, SAS, Cary, N.C.) Results are reported as the mean±standard deviation. Dunnet's test was used to make statistical comparisons between the two devices at each time point. A probability of p<0.05 was considered statistically significant. Curve fitting was performed using Excel (Excel 2010, Microsoft Corp).

Xience stents and BVS scaffolds were implanted in the coronary arteries of the same pigs. The arteries were then harvested 1, 3 6, 12, 18, 24, 30, 36, or 42 months later. The stent to artery expansion ratio was similar for each device type over the time course of the study. Expansion ratios were slightly higher for Xience than BVS (Table 2); this was because for Xience, the diameter of the expanded stent was measured whereas for BVS the diameter of the expanded balloon was measured as BVS is not radiopaque. In addition, 28 day EEL areas were similar for the two devices suggesting similar arterial expansion (Table 2). Despite these similarities, the arterial responses differed as noted below.

TABLE 2
Time course of changes in the epicardial coronary arteries and their properties after deployment of BVS or Xience.

| Time (months) | 1 | 3 | 6 | 12 | 18 | 24 | 30 | 36 | 42 |
|---|---|---|---|---|---|---|---|---|---|
| starting weight (kg) | 34.15 | 41.3 | 42.06 | 42.4 | 42.17 | 42.4 | 44.63 | 44.45 | 45.25 |
| stdev | 1.24 | 3 | 3.37 | 4.04 | 2.05 | 3.95 | 2.85 | 3.38 | 2.91 |
| Termination Weight (kg) | 41.06 | 64.16 | 68.09 | 85.47 | 92.06 | 99.2 | 100.45 | 105.98 | 104.18 |
| stdev | 1.91 | 5.61 | 3.61 | 4.6 | 4 | 5.4 | 6.5 | 4 | 8.1 |
| BVS Pre-implant MLD (mm) | 2.81 | 3 | 2.91 | 2.7 | 2.71 | 2.73 | 2.85 | 2.74 | 2.71 |
| stdev | 0.19 | 0.1 | 0.2 | 0.15 | 0.17 | 0.18 | 0.18 | 0.11 | 0.13 |
| BVS post-implant MLD (mm) | 2.89 | 3.11 | 2.98 | 2.78 | 2.84 | 2.81 | 2.95 | 2.81 | 2.8 |
| stdev | 0.22 | 0.1 | 0.16 | 0.19 | 0.18 | 0.2 | 0.15 | 0.1 | 0.11 |
| BVS follow-up MLD (mm) | 2.31 | 2.24 | 2.29 | 2.49 | 2.82 | 2.84 | 3.19 | 2.65 | 3.2 |
| stdev | 0.19 | 0.18 | 0.19 | 0.18 | 0.32 | 0.32 | 0.37 | 0.29 | 0.4 |
| Xience Pre-implant MLD (mm) | 2.77 | 2.85 | 2.86 | 2.68 | 2.63 | 2.67 | 2.87 | 2.79 | 2.69 |
| stdev | 0.19 | 0.12 | 0.21 | 0.16 | 0.07 | 0.09 | 0.2 | 0.18 | 0.15 |
| Xience post-implant MLD (mm) | 2.92 | 3.09 | 3.09 | 3.01 | 2.91 | 2.92 | 3.06 | 2.97 | 2.88 |
| stdev | 0.2 | 0.09 | 0.19 | 0.15 | 0.14 | 0.18 | 0.17 | 0.22 | 0.21 |
| Xience follow-up MLD (mm) | 2.67 | 2.34 | 2.71 | 2.77 | 2.78 | 3.03 | 3.08 | 2.58 | 3.05 |
| stdev | 0.23 | 0.42 | 0.26 | 0.23 | 0.13 | 0.26 | 0.32 | 0.32 | 0.29 |
| BVS Balloon to Artery ratio | 1.03 | 1.06 | 1.07 | 1.08 | 1.05 | 1.05 | 1.05 | 1.05 | 1.07 |
| stdev | 0.06 | 0.03 | 0.04 | 0.06 | 0.05 | 0.07 | 0.03 | 0.04 | 0.06 |
| Xience Balloon to Artery ratio | 1.11 | 1.12 | 1.1 | 1.15 | 1.19 | 1.13 | 1.11 | 1.11 | 1.14 |
| stdev | 0.06 | 0.05 | 0.04 | 0.05 | 0.06 | 0.02 | 0.05 | 0.03 | 0.04 |
| BVS EEL Area (mm2) | 8.08 | 8.28 | 8.29 | 9.54 | 10.66 | 10.82 | 12.33 | 11.56 | 12.22 |
| stdev | 1.16 | 0.81 | 0.8 | 1.93 | 1.51 | 1.75 | 1.54 | 1.63 | 1.51 |
| Xience EEL Area (mm2) | 7.86 | 9.31 | 8.49 | 8.72 | 8.41 | 9.16 | 9.49 | 8.29 | 8.34 |
| stdev | 0.98 | 2.26 | 1.02 | 1.18 | 0.7 | 1.06 | 1.01 | 1.13 | 0.71 |
| BVS Neointimal Area (mm2) | 1.864 | 2.387 | 2.494 | 2.408 | 2.489 | 2.741 | 2.974 | 2.773 | 2.624 |
| stdev | 0.452 | 0.703 | 0.446 | 0.893 | 0.388 | 0.54 | 0.613 | 0.535 | 0.358 |
| Xience Neointimal Area (mm2) | 0.909 | 2.267 | 1.758 | 1.288 | 1.124 | 1.241 | 1.352 | 1.481 | 1.152 |
| stdev | 0.257 | 1.498 | 0.307 | 0.564 | 0.401 | 0.46 | 0.607 | 0.743 | 0.205 |
| BVS Medial Area (mm2) | 1.347 | 1.555 | 1.139 | 1.425 | 1.166 | 1.547 | 1.327 | 0.847 | 1.297 |
| stdev | 0.265 | 0.42 | 0.247 | 0.344 | 0.338 | 0.561 | 0.305 | 0.351 | 0.348 |
| Xience medial area (mm2) | 1.103 | 2.253 | 1.052 | 1.198 | 1.021 | 1.471 | 1.098 | 0.715 | 1.093 |
| stdev | 0.096 | 2.059 | 0.214 | 0.388 | 0.133 | 0.367 | 0.213 | 0.293 | 0.198 |
| BVS wall thickness (mm) | 0.36 | 0.455 | 0.409 | 0.392 | 0.353 | 0.417 | 0.385 | 0.332 | 0.35 |
| stdev | 0.063 | 0.138 | 0.069 | 0.077 | 0.049 | 0.068 | 0.051 | 0.051 | 0.041 |
| Xience wall thickness (mm) | 0.218 | 0.501 | 0.3 | 0.257 | 0.226 | 0.276 | 0.244 | 0.233 | 0.237 |
| stdev | 0.033 | 0.374 | 0.037 | 0.083 | 0.041 | 0.072 | 0.064 | 0.064 | 0.031 |
| BVS circumferential wall stress (dynes/cm2) | 4.733 | 3.708 | 4.093 | 4.681 | 5.76 | 4.734 | 5.655 | 6.511 | 6.281 |
| stdev | 0.934 | 1.066 | 0.913 | 0.804 | 1.244 | 1.175 | 1.185 | 1.26 | 1.089 |
| Xience circumferential wall stress (dynes/cm2) | 8.499 | 4.902 | 6.048 | 7.852 | 8.635 | 7.406 | 8.703 | 8.51 | 7.96 |
| stdev | 1.42 | 2.673 | 0.862 | 2.009 | 1.866 | 2.268 | 2.305 | 2.394 | 1.316 |
| BVS Oct lumen area [mm2] | 4.25 | 4.34 | 4.58 | 5.33 | 6.19 | 6.73 | 8.11 | 6.93 | 8.77 |
| Stdev | 0.55 | 0.55 | 0.58 | 0.53 | 0.51 | 0.51 | 0.51 | 0.51 | 0.49 |
| Xience OCT lumen area (mm2) | 5.4 | 4.56 | 5.54 | 6.17 | 6.04 | 6.36 | 6.94 | 5.9 | 6.42 |
| Stdev | 0.46 | 0.4 | 0.43 | 0.43 | 0.43 | 0.43 | 0.4 | 0.4 | 0.4 |
| BVS wall shear stress (dynes/cm2) | 18.796 | 26.099 | 25.187 | 24.176 | 24.678 | 20.809 | 17.597 | 19.306 | 13.855 |
| stdev | 4.594 | 7.865 | 7.663 | 8.082 | 17.259 | 9.667 | 14.681 | 6.93 | 6.199 |
| Xience wall shear stress (dynes/cm2) | 13.011 | 31.517 | 18.653 | 18.334 | 19.784 | 20.41 | 18.09 | 23.958 | 19.905 |
| stdev | 2.78 | 22.094 | 5.215 | 5.464 | 2.682 | 6.16 | 6.477 | 6.698 | 3.078 |

Lumen Area Changes:

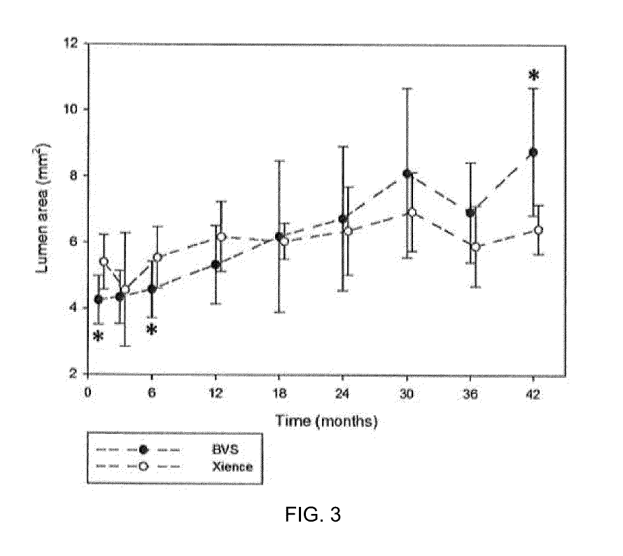

The OCT derived lumen areas of Xience-treated and BVS-treated arteries varied over the course of the study (Table 2, FIG. 3). Xience lumen area decreased progressively over the first 3 months post implant, gradually increased from 3 to 24 months, and then remained relatively flat thereafter. However, none of these changes were statistically significant.

In contrast, BVS lumen area decreased more quickly, reaching a nadir 1 month post-deployment with a lumen diameter significantly less than for Xience. Thereafter, there was a steady increase in BVS lumen area through 42 months with BVS lumen areas exceeding Xience lumen area at 30 months and beyond. At 42 months BVS lumen area was significantly greater than Xience lumen area (Xience 6.42+0.75 mm; BVS: 8.77+1.93 mm, p<0.01). Applying Poiseuille's equation, this difference in lumen area implies that the BVS-treated artery should be able to conduct 1.87-fold more blood flow than the Xience-treated artery.

Arterial Wall Changes:

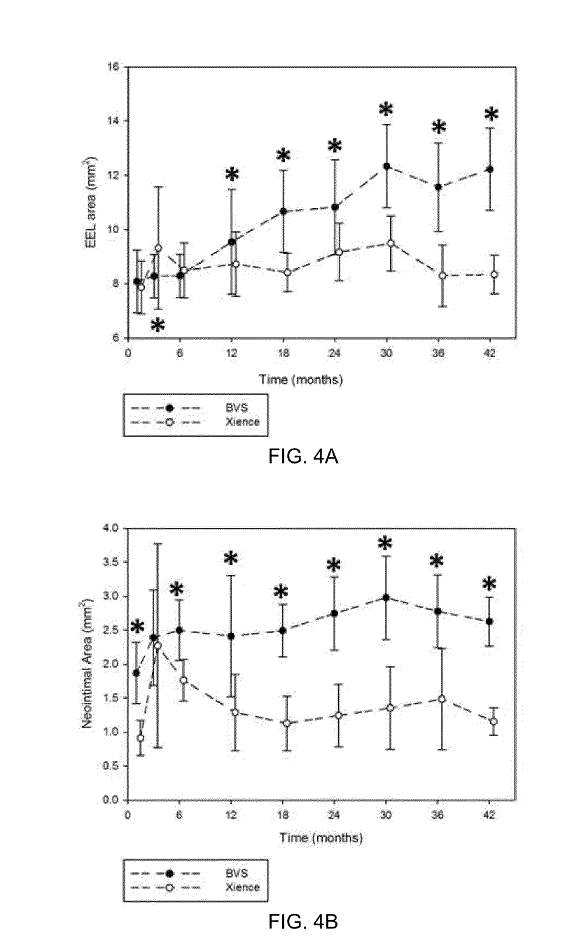

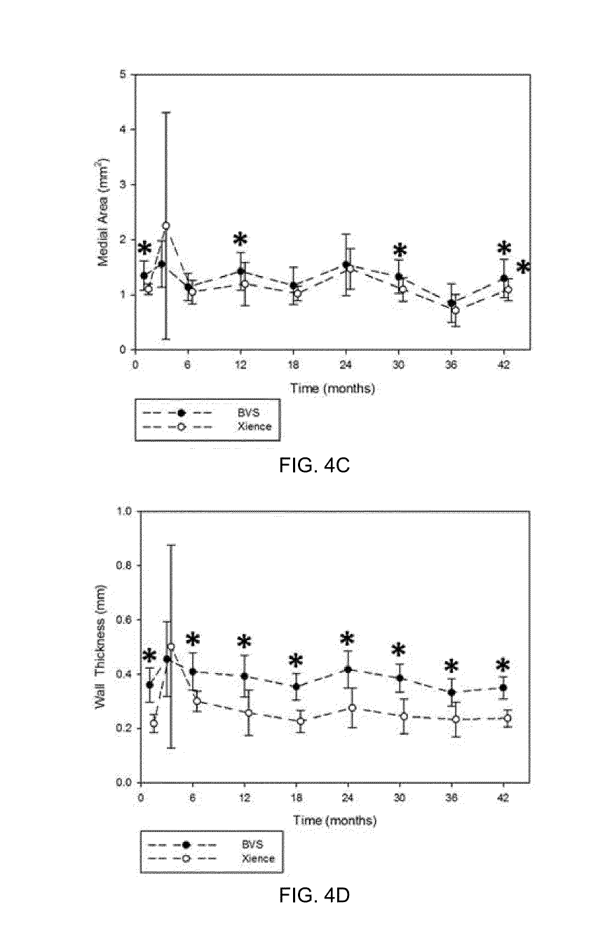

The changes in the arterial wall following device implant are summarized in Table 2 and FIGS. 4A-D. FIGS. 4A-D depict coronary artery histomorphometric changes as a function of time for BVS-treated and Xience-treated porcine coronary arteries. FIG. 4A depicts area circumscribed by the External Elastic Lamina (EEL) changes, FIG. 4B depicts neointimal area changes, FIG. 4C depicts medial area changes, and FIG. 4D depicts wall thickness changes.

For Xience-treated arteries, the area circumscribed by the EEL area remained relatively constant over the course of the study indicating that no remodeling had occurred. Neointimal area increased 1 and 3 months after stent implantation, regressed from 3 to 18 months, and then increased again from 18 to 36 months. Medial area followed a somewhat similar pattern with increases occurring 1 to 3 months post-implantation and a decrease occurring 3 to 6 months post-implantation. Paralleling the changes in intimal and medial thickness, wall thickness was increased 3 and 6 months compared to 1 month, but regressed back to the 1 month thickness 12 months and beyond.

In contrast, BVS-treated arteries underwent expansive remodeling with the area circumscribed by the EEL increasing progressively from 1 month to 30 months and then stabilizing. Neointimal area increased quickly in the first month after implantation and remained relatively stable thereafter. Medial area remained relatively stable over the course of the study. Consequently, wall thickness increased progressively over the first 3 months post-implantation and then remained relatively constant. Importantly, wall thickness was significantly greater for the BVS-treated arteries than the Xience-treated arteries at all time points except at 3 months.

Circumferential Wall Stress Changes:

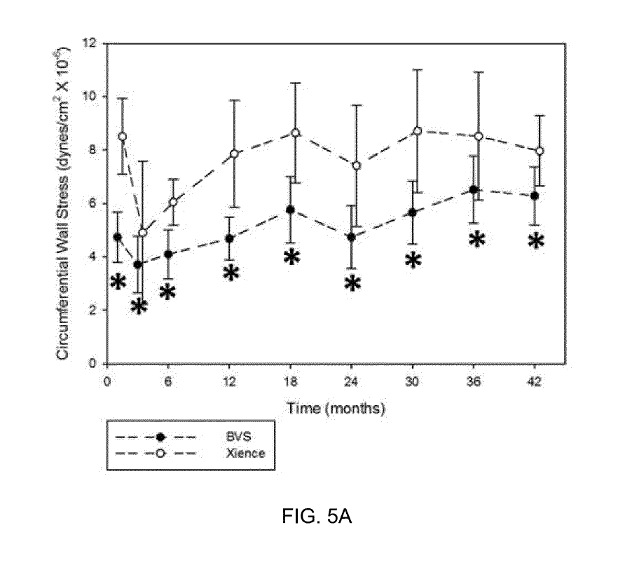

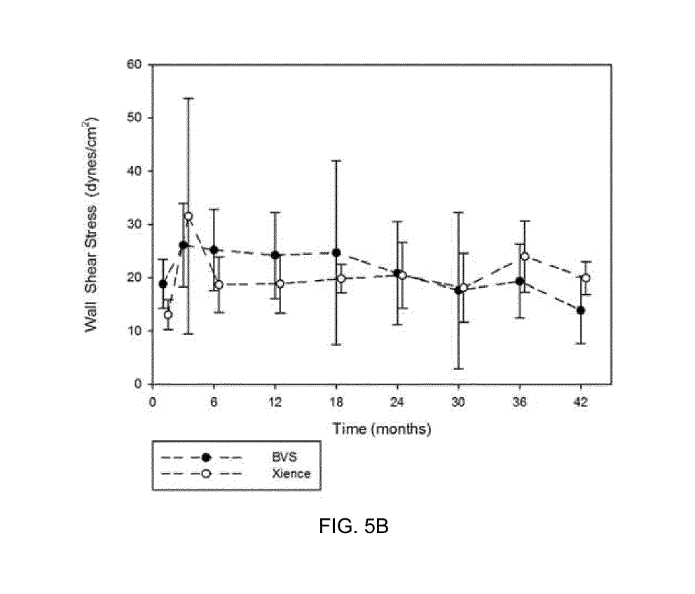

The changes in CWS as a function of time are summarized in Table 2 and FIG. 5A. For the Xience-treated arteries, CWS increased at the time of stent deployment. From 28 days to 3 months after deployment CWS decreased secondary to increases in wall thickness brought about by neointima formation and medial thickening. From 3 to 18 months, CWS gradually increased which was the result of decreases in both intimal area and medial areas that occurred over that time. Eighteen months and beyond, CWS remains relatively stable.

A somewhat similar response is seen for BVS-treated arteries. After the increase in CWS induced by BVS deployment, CWS decreased from 28 days to 3 months as neointimal area increased. Thereafter there was a gradual increase in CWS that resulted from a gradual increase in lumen area while wall thickness remained relatively constant. At all time points except 12 months, CWS in the BVS-treated arteries was less than the Xience-treated arteries presumably due to the presence of the Xience stent which would provide greater stress shielding of the artery.

Wall Shear Stress changes: In this study, pig weight at the time of sacrifice increased logarithmically (Table 2) (Weight=17.426 In (study time in months); R2=0.9873). This increase in body weight would be expected to increase cardiac output, cardiac work, coronary blood flow, and therefore increase coronary WSS, which would then stimulate flow-induced, expansive coronary artery remodeling that would return WSS to normal.

The changes in CWS as a function of time are summarized in Table 2 and FIG. 5B. As depicted in Table 2 and FIG. 5B, WSS in Xience-treated arteries increased from 1 month to 3 months after deployment, decreased over the next 3 months and then stayed relatively stable thereafter. Histopathologic analyses indicate that the changes in WSS at 3 and 6 months were due to changes in intimal and medial areas while the area circumscribed by the external elastic lamina remained unchanged.

In contrast, WSS in BVS-treated coronary arteries was significantly greater than in the Xience-treated arteries at 28 days (Table 2, FIG. 5B). WSS continued to increase and peak at 3 months following deployment. Thereafter, WSS progressively decreased. Histopathologic analyses indicate that the 1 month difference was due to greater neointimal and medial areas and wall thickness. Histopathologic analyses also indicate that the changes in WSS at 3 months and beyond were due to increases in the EEL area and lumen area (expansive remodeling) over this time. Overall, WSS was similar for both devices except for 28 days.

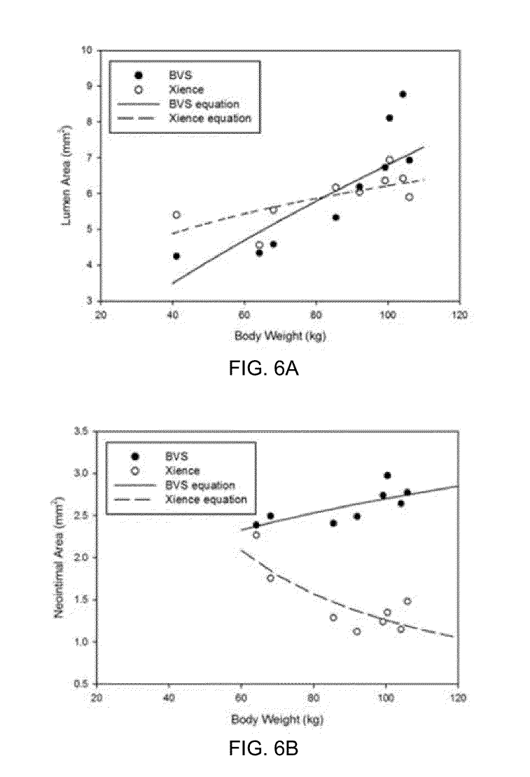

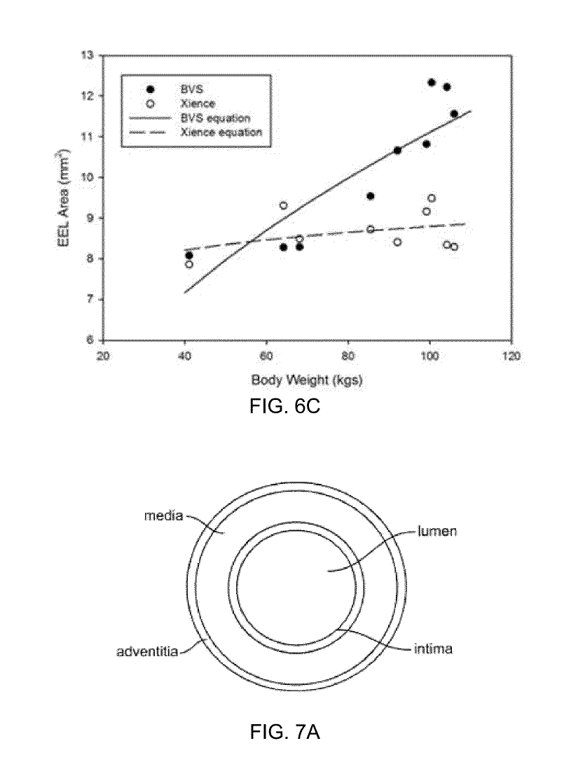

FIG. 6A depicts lumen area vs. body weight for BVS or Xience. To explore the mechanisms responsible for the maintenance of WSS, the lumen area vs. body weight data for Xience and BVS-treated arteries are fit to power curves:
Xience: Y=1.8444 X^{0.2563}, R²=0.4582
BVS: Y=0.2375 X^{0.3321}, R²=0.7286.
   This approach was based upon the fact that cardiac output and therefore coronary blood flow increases as function of body weight^{0.75}. [Holt 1968; West 1997; Weinberg 2007] The results (FIG. 6A) revealed that the exponent for BVS-treated arteries was greater than for Xience-treated arteries (exponents: Xience: 0.29; BVS 0.71) and close to the expected exponent 0.75, suggesting that the BVS-treated arteries undergo lumen area increases are more aligned with body weight increases.

FIG. 6B depicts lumen area vs. body weight for BVS or Xience. The neointimal area vs. body weight data for Xience and BVS-treated arteries over the 3 month to 42 month period are also fit to power curves:
Xience: Y=120.03 X^{-0.99}, R²=0.6529
BVS: Y=0.7064 X^{0.2912}, R²=0.5308.
   In this case, it was found that there was a negative exponent for Xience-treated arteries but a slightly positive exponent for the BVS-treated arteries (exponents: Xience: −0.99; BVS: 0.29) suggesting that Xience-treated arteries remodel through intimal thinning that inversely aligned with body weight.

FIG. 6C depicts EEL area vs. body weight for BVS or Xience. EEL area vs. body weight data are also fit to power curves:
Xience: Y=6.2334 X^{0.0747}, R²=0.143
BVS: Y=1.2264 X^{0.4797}, R²=0.7737.
   A positive exponent was found for BVS-treated arteries and an exponent close to zero for Xience-treated arteries suggesting that BVS-treated arteries undergo expansive remodeling whereas Xience-treated arteries do not.

The key finding of the study is that the BVS and Xience devices behave similarly in the short term but that in the mid-to-long-term BVS behaves differently. [Oberhauser J P, et al. Eurointervention 5:Suppl F: F15-F22, 2009]. In the short term, (revascularization phase), both devices serve as a scaffold to support an increased arterial diameter. This expansion stretches and injures the artery and increases CWS, changes that induce repair processes that increase arterial wall thickness and reduce lumen diameter and therefore reduce CWS. At the end of this phase, CWS has returned towards normal.

In the mid-term (restoration phase), Xience maintains its structural rigidity while the BVS begins to lose its radial strength. The EEL area of Xience-treated arteries remains unchanged over time indicating that no remodeling occurs.

In contrast, the EEL area of BVS-treated arteries expands over time demonstrating that these arteries can undergo the expansive remodeling that is needed to adjust for increases in WSS that occur as basal coronary flow increases to meet the demands of increased body weight. Since the Xience-treated arteries do not expand to control WSS, WSS appears to be controlled by remodeling and thinning of the neointima.

In the long term (remodeling phase), CWS and WSS stabilize in both the Xience-treated and BVS-treated arteries. However, at the end of this study, CWS was lower in the BVS treated arteries than in the Xience-treated arteries, presumably because the metallic stent shields the artery. WSS is approximately the same for both devices but the way in which this was achieved was different, i.e., by expansive remodeling in the BVS-treated arteries and by neointimal remodeling and thinning in the Xience-treated arteries.

A second finding of this study is that the Xience stents behaved similarly in this study as BMS and DES have behaved in other human and pig studies. After the initial increase in lumen area associated with the stent implant, there was a loss in area over the next 3 months as the neointima formed. From 3 months to 30 months, lumen area increased slightly as the neointima thinned. From 30 months to 42 months, lumen area remained relatively constant although there was an apparent dip at 36 months, possible the pigs studied at that time point were slightly smaller. A somewhat similar angiographic pattern of changes in lumen diameter was previously observed by Kimura T, et al. Circulation 105: 2986-2991, 2002 who found that, after stent placement in man, lumen diameter decreased through 6 months (restenotic phase), increased from 6 months to 3 years (regression phase), and then narrowed again beyond 4 years (renarrowing phase).

In Xience-treated arteries, a thinning or regression of the neointima was observed. Such regression has been observed for BMS in both man [Asakura M, et al., Circulation 97: 2003-2006, 1998] [Kimura 2002] and pig [Kim, W H, et al., Coronary Artery Disease 11: 273-277, 2000]. This thinning is, in part, the result of a maturation of the neointima which involves changes in proteoglycan composition (a replacement of versican with decorin and type I collagen with type III collagen) [Farb, A, et al. Circulation 110: 940-947, 2004]. It is also, in part, a response to control WSS as has been demonstrated in BMS [Wentzel 2001], DES [Papafaklis M I, et al. J Am Coll Cardiol Intv 3: 1181-1189, 2010], and vascular grafts [Mattsson E J R, et al. AVTB 1997; 17: 2245-2249].

There are a number of limitations to this study, most of which are related to the assumptions made in the calculation of CWS and WSS. The calculation of CWS assumed that arterial pressure was constant in all vessels at all times, which is unlikely. Nevertheless, since the BVS and Xience were implanted in the same pigs, any differences should cancel out. The calculation of WSS assumed that blood viscosity was constant and that blood flow was the same in all coronary arteries of each heart. Again, this should cancel out as the BVS and Xience were implanted in all arteries. Also for the WSS calculation, coronary blood flow was not measured; instead it was assumed that coronary flow increased as body weight increased. Previous research has documented that, in mammals, heart weight varies as the 0.98 power of body weight [Prothero J. Growth 43: 139-150, 1979]. However, metabolic rate and therefore cardiac output and coronary blood flow vary as body weight^{0.75} [Holt 1968] [West 1997]. Therefore, in this analysis, it was assumed that coronary blood flow was 5% of cardiac output which was a function of body weight^{0.75}. Coronary flow in each coronary artery was assumed to be one-third of total coronary flow.

A final limitation of this study is that the measurements were not obtained serially from the same pigs. Instead, the measurements were obtained from different groups of pigs studied at different times. This approach adds variability to the results and therefore makes it harder to discern trends and identify significant differences.

The results of this study may have potential clinical implications. In particular, in this study, lumen area of BVS-treated and Xience-treated arteries varied as 0.72 and 0.29 the power of body weight (FIGS. 6A, 6B) suggesting that BVS-treated arteries were better able to accommodate to increases in WSS that accompany increases in blood flow associated with increases in body weight. Similar increases in lumen area have been observed in pig studies evaluating other bioresorbable scaffolds [Strandberg E, et al., Circ Cardiovasc Intery 2012; 5: 39-46] [Durand E, et al., Circ Cardiovaasc Intery 2014; 7:70-79] and in the clinical evaluation of Absorb [Serruys P W, et al., Lancet 2009; 373: 897-910]. When lumen area at 42 months was used to determine blood flow conductance (BVS radius⁴/Xience radius⁴), it was found that the larger area in the BVS-treated arteries would provide 1.87 fold more blood flow than for the Xience-treated arteries.

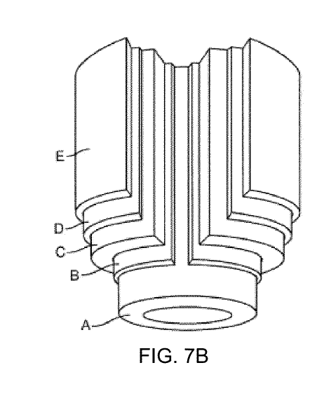

A wall of a healthy blood vessel is essentially made up of three distinct layers surrounding the lumen through which blood flows, the outermost advantitia, the media, and the intima. FIG. 7A depicts a radial cross-section of an artery and FIG. 7B depicts a section of an artery in three dimensions showing the intima (A), and the media (C), and the adventitia (E). The cells of the intima are supported by the internal elastic membrane or lamina (IEM or IEL) (B) that separates the intima from the media. The external elastic membrane or lamina (D) (EEM or EEL) is a concentration of elastic fibers at the inner boundary of the adventitia and the media.

The present invention is applicable to, but is not limited to, self-expandable stents, balloon-expandable stents, stent-grafts, and generally tubular medical devices in the treatment of artery disease. The present invention is further applicable to various stent designs including wire structures and woven mesh structures.

Self expandable or self expanding stents include a bioabsorbable polymer scaffold that expands to the target diameter upon removal of an external constraint. The self expanding scaffold returns to a baseline configuration (diameter) when an external constraint is removed. This external constraint could be applied with a sheath that is oriented over a compressed scaffold. The sheath is applied to the scaffold after the scaffold has been compressed by a crimping process. After the stent is positioned at the implant site, the sheath may be retracted by a mechanism that is available at the end of the catheter system and is operable by the physician. The self expanding bioabsorbable scaffold property is achieved by imposing elastic deformation to the scaffold during the manufacturing step that compresses the scaffold into the sheath.

The bioabsorbable scaffold may also be expanded by a balloon. In this embodiment the scaffold is plastically deformed during the manufacturing process to tightly compress the scaffold onto a balloon on a catheter system. The scaffold is deployed at the treatment site by inflation of the balloon. The balloon will induce areas of plastic stress in the bioabsorbable material to cause the scaffold to achieve and maintain the appropriate diameter on deployment.

Bioresorbable polymer scaffolds for coronary artery treatment can have a length between 8 to 48 mm. Such coronary scaffolds may be laser cut from polymer tubes with a diameter between 2.0 mm to 5.5 mm and with a thickness/width of 80-160 microns.

The coronary scaffold may be configured for being deployed by a non-compliant or semi-compliant balloon from about a 1.1 to 1.5 mm diameter (e.g., 1.35 mm) crimped profile. Exemplary balloon sizes include 2.5 mm, 3.0 mm, 3.5 mm, 4.0 and 4.5 mm, where the balloon size refers to a nominal inflated diameter of the balloon. The scaffold may be deployed to a diameter of between 2.5 mm and 5 mm, 2.5 to 4.5 mm, or any value between and including the endpoints. The pressure of the balloon to deploy the scaffold may be 7 to 30 psi. Embodiments of the invention include the scaffold in at a crimped diameter over and in contact with a deflated catheter balloon.

The intended deployment diameter may correspond to, but is not limited to, the nominal deployment diameter of a catheter balloon which is configured to expand the scaffold. The balloon pressure and the diameter to which the balloon inflates and expands the scaffold may vary from deployment to deployment. For example, the balloon may expand the scaffold in a range between the nominal inflated diameter to the nominal inflated diameter plus 0.5 mm, e.g., a 3.0 mm balloon may expand a scaffold between 3 and 3.5 mm. In any case, the inflated diameter at deployment is less than the rated burst diameter of the balloon.

A scaffold may be laser cut from a tube (i.e., a pre-cut tube) that is greater than or less than an intended deployment diameter. In this case, the pre-cut tube diameter may be 0.5 to 1.5 times the intended deployment diameter or any value or range in between and including the endpoints.

In a preferred embodiment a scaffold for coronary applications has the stent pattern described in U.S. application Ser. No. 12/447,758 (US 2010/0004735) to Yang & Jow, et al. Other examples of stent patterns suitable for PLLA are found in US 2008/0275537.

The bioresorbable stent may have a backbone, body, or scaffold that is PLLA-based, made of PLLA, a copolymer or blend of PLLA with another polymer or polymers. The polymer or polymers may be polycaprolactone, polyglycolide, polydioxanone, polytrimethylene carbonate, and poly(4-hydroxybutyrate). Other monomers that can be copolymerized with L-lactide to produce a copolymer are caprolactone, glycolide, dioxanone, and trimethylene carbonate.

When a bioresorbable scaffold is implanted, the mechanical properties (such as strength and modulus) and scaffold properties (such as radial strength, radial and axial stiffness) do not change for a period of time, even though the polymer is degrading. After this period, the mechanical and scaffold properties gradually change, for example, the strength, modulus, radial strength, radial stiffness gradually decrease.

The molecular weight of the scaffold decreases with time due to chain scission of the material by hydrolysis. Radial strength does not change for a period of time after implantation in spite of the decrease in molecular weight. However, after this period of time, the radial strength gradually decreases over a period of time. Mass loss is due to assimilation or dissolution of monomers and soluble oligomers resulting from hydrolysis of the polymer. Additionally, the loss of radial strength is followed by a gradual decline of mechanical integrity. The mechanical integrity loss refers to discontinuities in the scaffold struts.

The prevailing mechanism of degradation of many bioabsorbable polymers is chemical hydrolysis of the hydrolytically unstable backbone. In a bulk degrading polymer, the polymer is chemically degraded throughout the entire polymer volume. As the polymer degrades, the molecular weight decreases. The reduction in molecular weight results in changes in mechanical properties (e.g., strength) and stent properties. For example, the strength of the scaffold material and the radial strength of the scaffold are maintained for a period of time followed by a gradual or abrupt decrease. The decrease in radial strength is followed by a loss of mechanical integrity and then erosion or mass loss. Mechanical integrity loss is demonstrated by cracking and by fragmentation. Enzymatic attack and metabolization of the fragments occurs, resulting in a rapid loss of polymer mass.

The manufacturing process of a bioabsorbable scaffold includes selection of a bioabsorbable polymer raw material or resin. Detailed discussion of the manufacturing process of a bioabsorbable stent can be found elsewhere, e.g., U.S. Patent Publication No. 20070283552.

In general, a scaffold can be made of a bioresorbable aliphatic polyester. Additional exemplary biodegradable polymers for use with a bioabsorbable polymer scaffolding include poly(D-lactide) (PDLA), polymandelide (PM), polyglycolide (PGA), poly(L-lactide-co-D,L-lactide) (PLDLA), poly(D,L-lactide) (PDLLA), 96/4 poly(D,L-lactide) (PDLLA), poly(D,L-lactide-co-glycolide) (PLGA), poly(L-lactide-co-caprolactone), and poly(L-lactide-co-glycolide) (PLLGA). The poly(L-lactide-co-caprolactone) may have 1 to 5% (by mole or weight) of caprolactone.

With respect to PLLGA, the stent scaffolding can be made from PLLGA with a mole % of GA between 5-15 mol %. The PLLGA can have a mole % of (LA:GA) of 85:15 (or a range of 82:18 to 88:12), 95:5 (or a range of 93:7 to 97:3), or commercially available PLLGA products identified as being 85:15 or 95:5 PLLGA. The examples provided above are not the only polymers that may be used.

Polymers that are more flexible or that have a lower modulus than those mentioned above may also be used. Exemplary lower modulus bioabsorbable polymers include, polycaprolactone (PCL), poly(trimethylene carbonate) (PTMC), polydioxanone (PDO), poly(4-hydroxy butyrate) (PHB), and poly(butylene succinate) (PBS), and blends and copolymers thereof.

In exemplary embodiments, higher modulus polymers such as PLLA or PLLGA may be blended with lower modulus polymers or copolymers with PLLA or PLGA. The blended lower modulus polymers result in a blend that has a higher fracture toughness than the high modulus polymer. Exemplary low modulus copolymers include poly(L-lactide)-b-polycaprolactone (PLLA-b-PCL) or poly(L-lactide)-co-polycaprolactone (PLLA-co-PCL). The composition of the blend can include 1-5 wt % of low modulus polymer.

A scaffold may also be made from a tyrosine-derived polycarbonate. These degradable polymers are derived from the polymerization of desaminotyrosyl-tyrosine alkyl esters. J. of Appl. Polymer Sci., Vol. 63, 11, pp. 1467-1479. In the synthesis of tyrosine-derived polycarbonates, L-tyrosine and its natural metabolite desamino-tyrosine [3-(4-hydroxphenyl) propionic acid] are used as building blocks to form desaminotyrosyl-tyrosine alkyl esters. Exemplary tyrosine-derived polycarbonates include poly(DTE carbonate), poly(DTB carbonate), poly(DTH carbonate), poly(DTO carbonate), and poly(DTBzl carbonate), where “DT” refers to desamino-tyrosyl-tyrosine and “E,” “B,” “H,”, “O,” and “Bzl” refer to ethyl, butyl, hexyl, octyl, and benzyl esters, respectively.

The bioresorbable scaffold may also be made from poly-anhydride ester. The polyanhydrides ester may be based on salicylic acid and adipic acid anhydride. The bioresorbable scaffold may be made from bioerodible metals or metal alloys including magnesium, iron, zinc, tungsten, and alloys including these metals. A durable or non-degradable stent may be made metals including platinum, stainless steel, and nickel-titanium alloys.

The BVS scaffolds may be coated with a polymer mixture that includes an therapeutic agent. The therapeutic agent may be an antiproliferative agent, anti-inflammatory agent, or both. In general, the anti-proliferative agent can be a natural proteineous agent such as a cytotoxin or a synthetic molecule or other substances such as actinomycin D, or derivatives and analogs thereof (manufactured by Sigma-Aldrich 1001 West Saint Paul Avenue, Milwaukee, Wis. 53233; or COSMEGEN available from Merck) (synonyms of actinomycin D include dactinomycin, actinomycin IV, actinomycin actinomycin X1, and actinomycin C1), all taxoids such as taxols, docetaxel, and paclitaxel, paclitaxel derivatives, all olimus drugs such as macrolide antibiotics, rapamycin, novolimus, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Representative rapamycin derivatives include 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin (ABT-578 manufactured by Abbott Laboratories, Abbott Park, Ill.), prodrugs thereof, co-drugs thereof, and combinations thereof.

These agents can also have anti-proliferative and/or anti-inflammatory properties or can have other properties such as antineoplastic, antiplatelet, anti-coagulant, anti-fibrin, antithrombonic, antimitotic, antibiotic, antiallergic, antioxidant as well as cystostatic agents.

“Molecular weight” can refer to number average molecular weight (Mn) or weight average molecular weight (Mw). Molecular weight values may refer to that obtained from Gas Permeation Chromotography using polystyrene reference standards.

The “glass transition temperature,” Tg, is the temperature at which the amorphous domains of a polymer change from a brittle vitreous state to a solid deformable or ductile state at atmospheric pressure. In other words, the Tg corresponds to the temperature where the onset of segmental motion in the chains of the polymer occurs. When an amorphous or semi-crystalline polymer is exposed to an increasing temperature, the coefficient of expansion and the heat capacity of the polymer both increase as the temperature is raised, indicating increased molecular motion. As the temperature is increased, the heat capacity increases. The increasing heat capacity corresponds to an increase in heat dissipation through molecular movement. Tg of a given polymer can be dependent on the heating rate and can be influenced by the thermal history of the polymer as well as its degree of crystallinity. Furthermore, the chemical structure of the polymer heavily influences the glass transition by affecting mobility.

The Tg can be determined as the approximate midpoint of a temperature range over which the glass transition takes place. [ASTM D883-90]. The most frequently used definition of Tg uses the energy release on heating in differential scanning calorimetry (DSC). As used herein, the Tg refers to a glass transition temperature as measured by differential scanning calorimetry (DSC) at a 20° C./min heating rate.

“Stress” refers to force per unit area, as in the force acting through a small area within a plane. Stress can be divided into components, normal and parallel to the plane, called normal stress and shear stress, respectively. Tensile stress, for example, is a normal component of stress applied that leads to expansion (increase in length). In addition, compressive stress is a normal component of stress applied to materials resulting in their compaction (decrease in length). Stress may result in deformation of a material, which refers to a change in length. “Expansion” or “compression” may be defined as the increase or decrease in length of a sample of material when the sample is subjected to stress.

“Strain” refers to the amount of expansion or compression that occurs in a material at a given stress or load. Strain may be expressed as a fraction or percentage of the original length, i.e., the change in length divided by the original length. Strain, therefore, is positive for expansion and negative for compression.

“Strength” refers to the maximum stress along an axis which a material will withstand prior to fracture. The ultimate strength is calculated from the maximum load applied during the test divided by the original cross-sectional area.

“Modulus” may be defined as the ratio of a component of stress or force per unit area applied to a material divided by the strain along an axis of applied force that results from the applied force. The modulus typically is the initial slope of a stress-strain curve at low strain in the linear region.

The present invention includes any combination of the embodiments or claims disclosed herein.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A method of treating coronary artery disease (CAD) and a cardiac disease that stimulates enlargement of the epicardial artery with a bioresorbable scaffold in a patient in need thereof comprising:
implanting the bioresorbable stent at stenotic lesion in a segment of an epicardial artery of the patient which increases a diameter of the stenotic segment of the artery,
wherein the patient has a cardiac disease that stimulates enlargement of the epicardial artery resulting from an increase in basal blood flow,
wherein the bioresorbable stent is configured to support the segment at the increased diameter to treat the stenosis and restore freedom of radial movement of the segment through a decrease in radial strength, loss of mechanical integrity, and resorption of the bioresorbable stent, and
wherein the restoration of freedom of radial movement of the segment provides, enables, permits, or no longer restrains expansive remodeling of the segment required by the increase in basal blood flow.

2. The method of claim 1, wherein the cardiac disease is selected from the group consisting of dilated cardiomyopathy, hypertrophic cardiomyopathy, aortic valve disease, mitral valve regurgitation, thyroid disease, left ventricular hypertrophy, anemia, or any other disease that increases coronary blood flow.

3. The method of claim 1, wherein the cardiac disease is left ventricular hypertrophy.

4. The method of claim 1, wherein the cardiac disease is dilated cardiomyopathy, wherein the patient has an enlarged heart cavity.

5. The method of claim 4, wherein the patient has Barth syndrome which caused the dilated cardiomyopathy.

6. The method of claim 1, wherein the cardiac disease is hypertrophic cardiomyopathy and a muscle mass of the patient's left ventricle is enlarged.

7. The method of claim 6, wherein the hypertrophic cardiomyopathy is hypertrophic obstructive cardiomyopathy, a septum between the a left and right heart ventricles of the patient is enlarged and obstructs blood flow from the left ventricle.

8. The method of claim 7, wherein the enlarged septum distorts one leaflet of a mitral valve of the patient causing leakage from the mitral valve.

9. The method of claim 1, wherein the cardiac disease is aortic valve disease.

10. The method of claim 9, wherein the aortic valve disease is aortic regurgitation, wherein the patient's aortic valve is widened or weakened which causes blood to flow backward from into the patient's left ventricle.

11. The method of claim 10, wherein the patient has holes in leaflets of the aortic valve which causes the aortic regurgitation.

12. The method of claim 10, wherein the aortic regurgitation is caused by a condition selected from the group consisting of rheumatic fever, a bicuspid aortic valve, a high blood pressure including a diastolic pressure more than 110 mm Hg, Marfan syndrome, endocarditis, ankylosing spondylitis, and dissecting aortic aneurysm.

13. The method of claim 9, wherein the aortic valve disease comprises aortic stenosis comprising a narrowing or blockage of the aortic valve of the patient.

14. The method of claim 13, wherein leaflets of the aortic valve are coated with deposits that alter a shape of the leaflets and reduce blood flow through the valve.

15. The method of claim 1, wherein the cardiac disease is mitral valve regurgitation in which the mitral valve of the patient allows leaking of blood backward into the patient's heart due to damage to the valve that prevents tight closure of the valve.

16. The method of claim 15, wherein the mitral valve regurgitation is chronic.

17. The method of claim 16, wherein the mitral valve regurgitation is caused by a condition selected from the group consisting of heart failure, rheumatic fever, congenital heart disease, and calcium buildup in the valve.

18. The method of claim 15, wherein the mitral valve regurgitation is acute.

19. The method of claim 18, wherein the acute mitral valve regurgitation is due to rupture of the mitral valve or nearby tissue.

20. The method of claim 18, wherein the acute mitral valve regurgitation is due to heart attack or endocarditis.

21. The method of claim 1, wherein the cardiac disease is hyperthyroidism.

22. The method of claim 1, wherein the cardiac disease is anemia.

23. A method of treating coronary artery disease (CAD) and an event that precipitates enlargement of an epicardial artery with a bioresorbable scaffold in a patient in need thereof comprising:
implanting the bioresorbable stent at a stenotic lesion in a segment of the epicardial artery of the patient,
wherein the patient has experienced an event that precipitates enlargement of the epicardial artery resulting from an increase in basal blood flow,
wherein the bioresorbable stent is configured to support the segment at the increased diameter to treat the stenosis and restore freedom of radial movement of the segment through a decrease in radial strength, loss of mechanical integrity, and resorption of the bioresorbable stent, and
wherein the restoration of freedom of radial movement of the segment providing expansive remodeling of the segment required by the increase in basal blood flow.

24. The method of claim 23, wherein the event is a myocardial infarction.

25. The method of claim 23, wherein the event is damage to a mitral valve.

26. A method of treating coronary artery disease (CAD) and a condition that stimulates enlargement of the epicardial artery with a bioresorbable scaffold in a patient in need thereof comprising:
reducing or eliminating stenosis with the bioresorbable stent at a stenotic lesion in a segment of an epicardial artery of the patient,
wherein the patient has a cardiac disease that stimulates enlargement of the epicardial artery resulting from an increase in basal blood flow, has experienced an event that precipitates enlargement of the epicardial artery resulting from an increase in basal blood flow, or is engaged in an endurance exercise program that stimulates enlargement of the epicardial artery resulting from an increase in basal blood flow; restoring freedom of radial movement of the segment that was restricted during the reduction or elimination of stenosis; and
causing enlargement of the segment of the epicardial artery resulting from an increase in basal blood flow due to the cardiac disease, event, or exercise program.
